(19) United States
(12) Patent Application Publication
(10) Pub. No.: US 2003/0120324 A1
(43) Pub. Date: Jun. 26, 2003

(54) System and method for remote programming of a medical device

(76) Inventors: Brett A. Osborn, New York, NY (US); Brian H. Kopell, New York, NY (US)

Correspondence Address: Jonathan S. Caplan, Esq., Kramer Levin Naftalis & Frankel LLP, 919 Third Avenue, New York, NY, 10022, US

(21) Appl. No.: 10/033,250

(22) Filed: Dec. 26, 2001

(51) Int. Cl. A61N 1/18

(52) U.S. Cl. 607/60

(57) ABSTRACT

A system and method allows a physician to remotely change the settings of a medical device that is implanted in a patient who is physically separated from the patient. The physician uses a computer to remotely access the patient's computer and programs new settings for the medical device that is conveyed to an emulator which in turn conveys the settings to the medical device. Patients who have neurostimulators implanted in them are particularly suited to receive the benefits of the present invention.

### FIELD OF THE INVENTION

The present invention relates to a system and method for remotely programming medical devices. More particularly, the system and method can be used for remotely programming an implanted neurostimulator.

### BACKGROUND INFORMATION

Recent developments in telecommunications have spawned new advancements in other fields. One field that has particularly benefited from the Internet revolution is medicine. Medical patients have been introduced to diverse applications such as telemedicine and online pharmacies. These applications have access to medical personnel even though patients are remotely located from the personnel.

Many patients have medical devices implanted within their bodies to regulate or facilitate bodily functions. Some of these devices, especially electronic devices, require the patient to periodically visit a medical practitioner in order for the practitioner to adjust or change the device's settings. For some of these patients, travel to and from their home to the office of their clinicians may be physically challenging or expensive. Thus, it is desirable to have a system and method that allow a medical practitioner to remotely program a medical device that is located with the patient. It is even more desirable to have a system that allows the remote programming to be accomplished with readily accessible means or with minimal special equipment.

### SUMMARY OF THE INVENTION

According to an exemplary embodiment of the present invention, a system and method are provided allowing a medical device to be remotely programmed or adjusted by a programmer who is physically separated from the medical device. For example, such a system and method can be used with a medical device that has been implanted in a patient. A physician or other suitably trained individual, using the system and method, can remotely program the implanted medical device through any type of communications channel, for example, the Internet. The medical device can also transmit its current and revised settings through the same communications channel.

### BRIEF DESCRIPTION OF THE DRAWINGS

FIG. 1 is a schematic representation of a conventional prior art implanted medical device, for example a neurostimulator, having a console programmer.

FIG. 2 is a block diagram showing the system architecture of an exemplary embodiment of the remote programming system according to the present invention.

FIG. 3 is a block diagram showing the system located at the patient for use in an exemplary embodiment of the remote programming system.

FIG. 4 is a flow diagram depicting a method of using the remote programming system according to an exemplary embodiment of the remote programming system.

FIG. 5 is a flow diagram illustrating a review sequence of the remote programming system according to an exemplary embodiment of the remote programming system.

FIG. 6 is a flow diagram illustrating a programming sequence of the remote programming system according to an exemplary embodiment of the remote programming system.

FIG. 7 is an exemplary screen shot of a computer screen a programmer would view when using the system of the present invention.

### DETAILED DESCRIPTION

The present invention features a system and method that allow a physician, or other medical practitioner, to remotely access a medical device in order to program the device, adjust the settings of the device, or monitor the parameters of the device. This system can be used with any type of medical device that is capable of electronically communicating with a computer or the like.

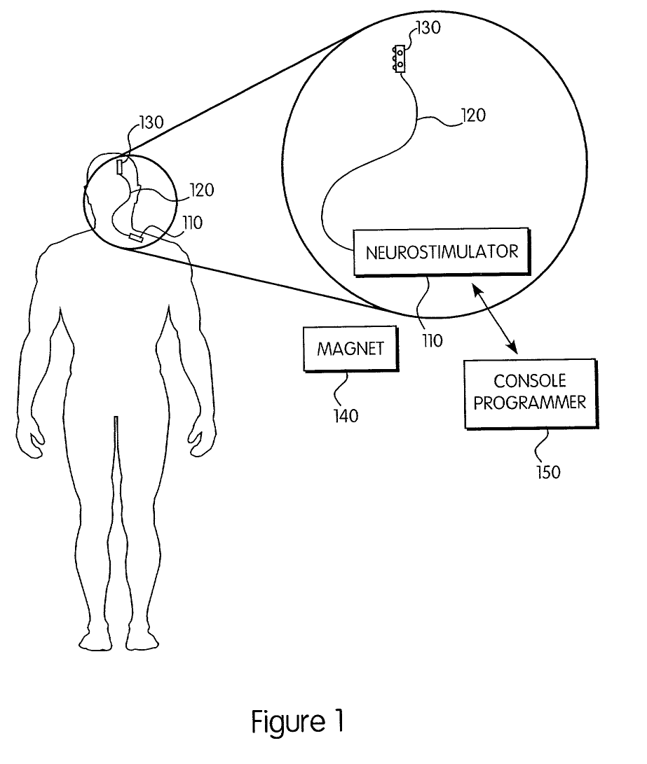

FIG. 1 illustrates an example of a conventional medical device that can be used with an exemplary embodiment of the remote programming system. Examples of medical devices include, but are not limited to, cardiac pacemakers, implantable cardioverter defibrillators, infusion pumps and artificial hearts. Specifically, in FIG. 1, a tremor control system 100 is shown as an example. Any type of electronic device or implant, however, can be used in conjunction with the remote programming system according to an embodiment of the present invention. Tremor control system 100 includes, for example, a neurostimulator 110, a lead 120, electrodes 130, a control magnet 140 and a console programmer 150.

Tremor control system 100 can be used in the treatment of patients that suffer from tremors due to diseases such as Parkinson's disease and Essential Tremor. As is known in the art, tremor control system 100 creates electrical stimulation in a patient's subthalamus or thalamus in order to block the brain signals that cause the tremors. An example of a tremor control system 100 that is commercially available and that can be used with an exemplary embodiment of the present invention is the ACTIVA® system available from Medtronic Inc. of Minneapolis, Minn.

As shown in FIG. 1, neurostimulator 110 is implanted near a patient's collarbone and is responsible for generating electrical pulses that block the brain signals. Lead 120 can be a thin wire that conducts the electrical pulses from the neurostimulator 110 to the electrodes 130 which are located in the patient's subthalamus or thalamus. For example, the lead 120 can connect to five separate electrodes 130. As is known in the art, each electrode 130 can be programmed to have a positive value, negative value or no value.

Control magnet 140, which is not implanted in the body, serves, for example, as a noninvasive modulator of the tremor control system 100. The console programmer 150 also can is be an external component and which is used by a physician or trained medical staff to adjust the parameters of the neurostimulator 110 via a communications link with the neurostimulator. The communications link between the console programmer 150 and the neurostimulator 110 is noninvasive, such as a radio frequency (RF) or infrared (IR) link from a transceiver component coupled to the console programmer 150. An example of an RF link between an external programmer and an implanted medical device (e.g., pacemaker) is described in U.S. Pat. No. 4,550,370.

Conventionally, once the neurostimulator 110 is implanted, the patient travels to a physician's office or hospital periodically so that the physician can evaluate the condition of the patient and make any required changes to the settings of the tremor control system 100. For some patients, the travel to and from the physician's office can be arduous. Typically, most practitioners capable of diagnosis, such as neurologists, have their medical practices in major metropolitan areas whereas many patients reside in the suburbs of metropolitan areas or even further from their practitioner.

The present invention eliminates the need for the patient to physically travel to the location of the physician by providing a system that enables the physician to remotely diagnose the patient and adjust the settings of the medical device as necessary.

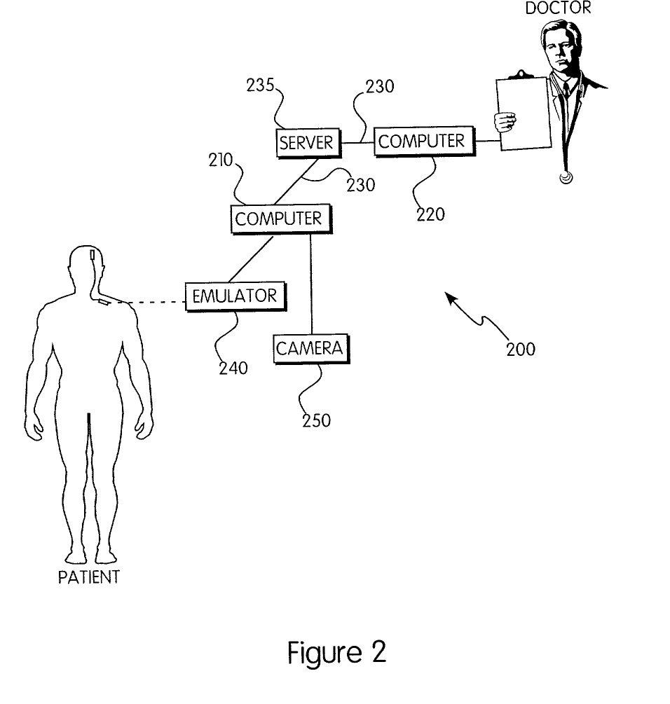

FIG. 2 shows a basic overview of the components in an exemplary embodiment of a remote programming system 200 that allows a physician to electronically communicate with a patient's medical device even though the physician and the patient are physically isolated from each other. Remote programming system 200 includes, for example, a patient's computer 210 in communication with a physician's computer 220. The computers 210, 220 can be in communication through any type of appropriate data communications medium 230 such as the Internet. In the case of the Internet, the computers 210 and 220 will typically communicate with each other via one or more servers 235. Alternatively, the computers 210 and 220 can be directly connected through a communications link such as a telephone line or T1 trunk. In another exemplary embodiment, the interconnection of the computers 210 and 220 may include wireless means such as cellular or satellite links. In yet another exemplary embodiment, the patient's computer 210 or the physician's computer 220 can include a special purpose device (e.g., a dummy terminal) or limited function computing device such as a personal digital assistant or hand-held device having the ability to establish a communication link with the other computer.

Patient's computer 210 is connected to an emulator 240, which may be internal or external to the computer 210, and a feedback component 250. Emulator 240, for example, mimics the functions of a console programmer used by the physician to adjust the tremor control system 100 traditionally done with prior art systems even though emulator 240 is now physically separated from the physician. For example, the console programmer of the ACTIVA® Tremor Control System, the Medtronic 7432 Neurological Programmer, uses bursts of RF signals at a frequency of 175 kHz to communicate with the neurostimulator. Pulse intervals of the bursts represent logic ones and zeroes that allow the RF signals to be transduced into digital signals and vice versa. The pulse intervals for a logic one and zero can be about 1775 μs and 450 μs, respectively. Emulator 240 provides the control signals to a suitable transceiver component for such RF signals at pulse intervals that make up thirty-two bits in length to form “words” that convey data between the emulator 240 (e.g., via the transceiver component) and the medical device such as neurostimulator 110. Emulator 240 can use AC or DC power.

Naturally, the specifics of the interface and communications protocol between the emulator 240 and the implanted device are not material to the present invention, so long as the emulator 240 and implanted device are compatible and adhere to the same protocol. Various interfaces and protocols are known in the art and need not be described further for purposes of the present invention.

The feedback component 250 may include, for example, a device that is able to perceive or detect the condition of the patient and convey that condition to the physician. For example, the feedback component 250 can be a video camera positioned to provide a view of the patient. In the case of a system for controlling tremors, for instance, the camera is able to capture and communicate images of the patient's dyskinesias and/or tremors through the patient's computer 210 to the physician's computer 220. Instead of visual images, the feedback component 250 can capture other types of signals through, for example, suitable biosensors, that are appropriate for other senses, for example audio signals, or the degree of rigidity in the patient's arms or legs.

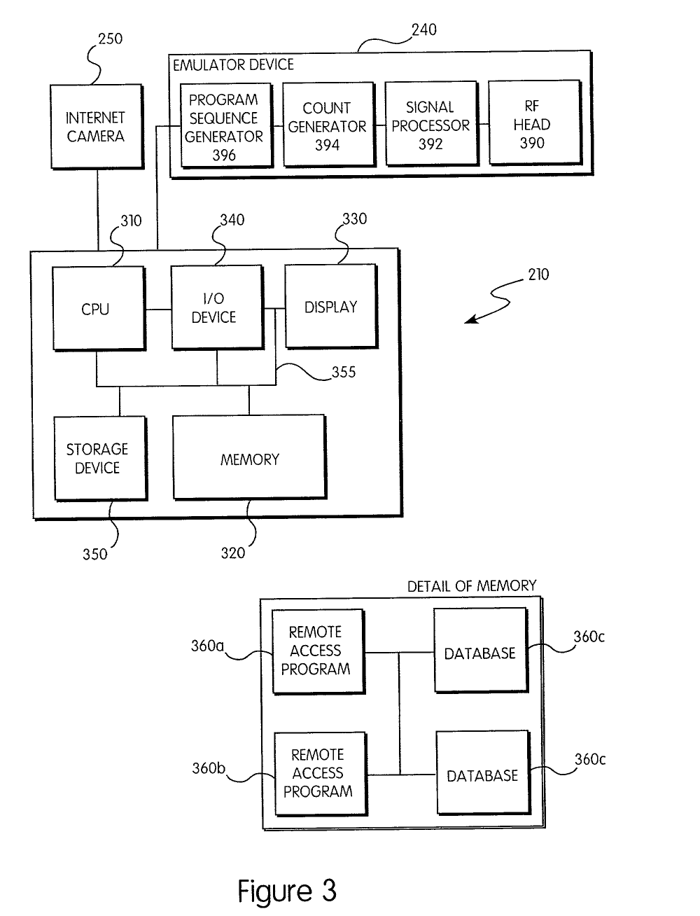

FIG. 3 is a block diagram of an exemplary embodiment of the remote programming system 200 on the patient's end. For example, the patient's computer 210 includes, for example, a central processing unit 310, random access memory 320, a display 330, input/output device(s) 340, and a storage device 350. The components of the patient's computer 210 are coupled, for example, via a conventional bus 355. Storage device 350 contains various modules 360 used to implement an exemplary embodiment of the present invention. For example, modules 360a, 360b, 360c respectively represent a remote access program, an emulator program, and a database. These modules can be separate programs and applications or a single program and application written in conventional programming language such as C++, Visual BASIC 6.0 or JAVA. The database, for example, can store communications protocol for multiple medical devices or multiple models of the same medical device. The patient's computer 210 may be implemented, for example, with a conventional personal computer (PC), workstation or the like.

Also connected to patient's computer 210 is feedback component 250 and emulator 240. As discussed above, the feedback component 250 can be a camera, such as a web cam, or the like. Preferably, the camera should be able to capture and the system should be able to process and convey real-time video of 640×480 pixels at thirty frames per second.

As described above, the emulator 240 is able to transduce electrical signals into a signal compatible with the medical device. In an exemplary embodiment of the present invention, the emulator 240 includes an RF head 390, a signal processor 392, a count generator 394 and a program sequence generator 396. RF head 390 is able to transmit and receive RF signals with a medical device such as an implanted neurostimulator and may be a separate component connected by a cable to the other components of emulator 240, thereby allowing manipulation of the RF head 390 by the patient (e.g., to place the RF head 390 near the implanted medical device).

Signal processor 392 receives, for example, incoming analog waveforms from RF head 390 (e.g., transmitted from the medical device) and, for example, amplifies the signal (e.g., with a gain of 1,000,000) and integrates the waveform to generate an approximate square wave. Noise is subsequently removed from the waveform, thus generating a true square waveform received from the medical device.

Count generator 394 receives, for example, input from the signal processor 392 and generates “counts” under, for example, every rising edge of the waveform. These counts are transmitted to the CPU 310 whereby the CPU 310 converts the waveform into a binary format. This binary format enables the CPU 310 to interpret the waveform.

Program sequence generator 396 receives binary data from the CPU 310 (e.g., instructions to alter parameters of the medical device) and converts the data into, for example, a pulse-interval modulated square wave output which is fed, for example, into a buffer and subsequently into the RF head 390 for transmission to the medical device in a known manner (e.g., using the appropriate protocol for the medical device). For example, the output from the program sequence generator 396 can be derived from look-up tables stored in the memory of computer 210 using the binary values received for particular parameter values.

The emulator 240 can be connected to the patient's computer 210 via any standard connection, for example, a serial or parallel port or a PCI interface.

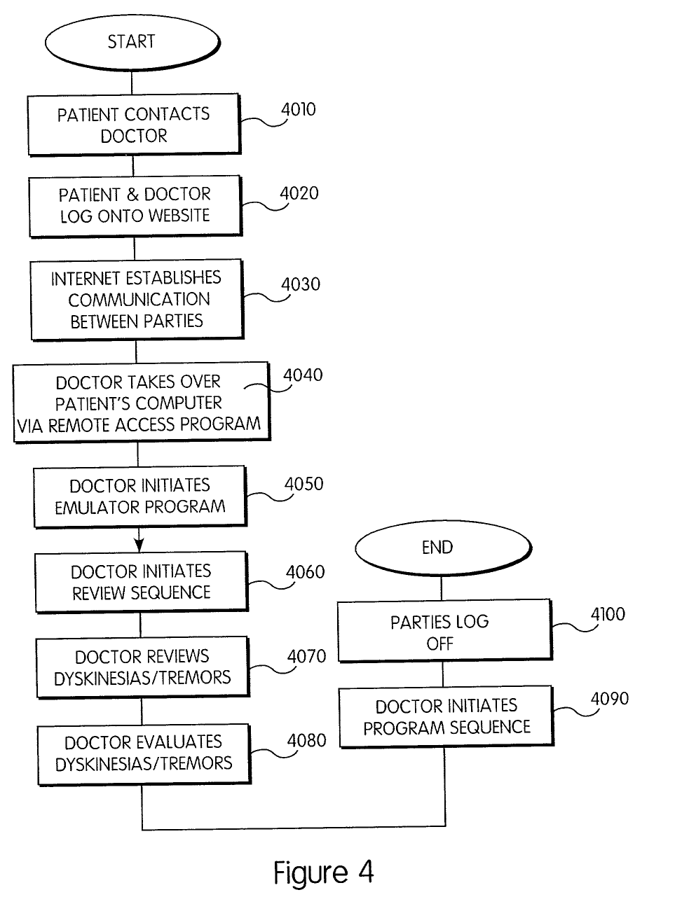

FIG. 4 depicts a flow diagram illustrating an exemplary embodiment of a method of using the remote programming system to adjust or change the parameters of the implanted medical device.

At 4010, contact between the physician and patient is initiated by any suitable means. For example, the patient may have scheduled an appointment with the physician to have the parameters of the medical device re-adjusted. Alternatively, the patient may be experiencing an emergency and needs medical attention as soon as possible.

At 4020, both the physician and patient log onto their respective computers.

At 4030, the computers are placed in communication with each other, for example, through a direct connection or with an intermediary such as a server as used with the Internet. For example, the physician and patient can facilitate their communication with video and/or chat technology as are known in the art.

At 4040, using computer 220, the physician remotely accesses a remote access program residing in the memory of the patient's computer 210. This remote access program allows the physician to gain access, or effectively take control of the patient's computer 210. Several suitable remote access programs are commercially available such as, for example, PcAnywhere from Symantec of Cupertino, Calif. or Netmeeting from Microsoft Corp. of Redmond, Wash.

At 4050, the physician accesses an emulator program residing in the memory of the patient's computer 210. The emulator program is responsible for generating the necessary signals to modify the settings of the medical device. The emulator program, in essence, transforms the patient's general purpose computer into a device comparable to the console programmer 150 that the physician would have used to program the medical device as if the patient were in physical proximity to the physician. The emulator program, for example, can access database 360c to retrieve the respective communications protocol for the medical device. The emulator program can also perform a check to ensure that the communications protocol being used matches the patient's medical device.

The emulator program should be secure, such that the patient, or any other non-medical or unqualified person, cannot access the program and change the settings of the medical device. Various means known in the art can be used to implement such security. For example, one method is to implement password protection that prevents access by those lacking knowledge of the password. An alternative method may be to load the emulator program into a separate secure server instead of the patient's computer. In this configuration, the computers of the physician and patient would be in electronic communication with the secure server. For example, only the physician would be allowed access to the emulator program residing on the server. If this method were used, care must be taken to ensure that the data being transmitted from the server to the patient's computer is not lost or corrupted. In yet a further embodiment, the physician's computer 220 may act as the server.

At 4060, the physician initiates, for example, a review sequence using the emulator program. This review sequence is described in greater detail below. The purpose of the review sequence is, for example, to apprise the physician of the current settings of the medical device.

At 4070, the patient uses the feedback component 250 connected to the patient's computer to transmit visual or sensory data related to the patient's current condition. For example, if the patient is experiencing tremors, a camera can be focused on the patient and images of the tremors can be transmitted to the physician. If biosensors are being used, they could be placed on the respective body part of the patient and coupled to computer 210 to transmit the patient's biosensor data.

At 4080, the physician can enhance the diagnosis of the condition of the patient by viewing the transmitted images or biosensor data. From this, the physician can determine which parameters should be changed to implement the treatment.

At 4090, the physician initiates the program sequence using the emulator program. The program sequence conveys the changes to the emulator which in turn conveys the settings to the medical device. This program sequence is discussed in greater detail below.

At 4100, both parties log-off their respective computers, and the remote programming of the medical device concludes.

In an exemplary embodiment of the present invention, all of the changes and actions made by the physician can be saved to databases located within the physician's and/or patient's computer. Saving this information to the database(s) also creates a record of the session. These records can be accessed in the future in order to generate a history of the patient's treatments, for example, for the primary care neurologist or any outside neurologists or other medical professionals to access for research purposes and to potentially improve the care of future patients.

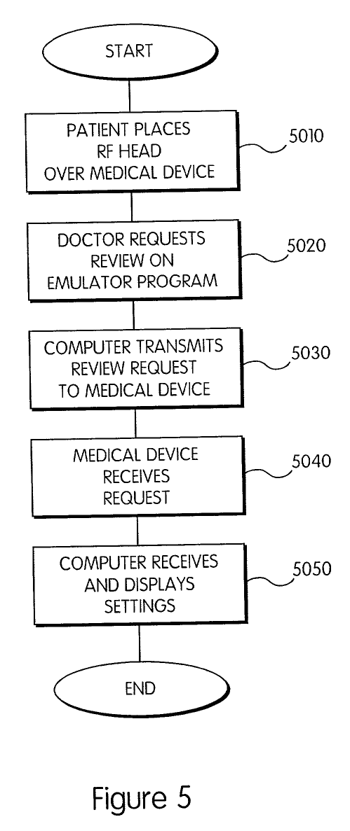

FIG. 5 depicts an exemplary embodiment of the review sequence mentioned above. At 5010, the patient places emulator 240 near the medical device so that the emulator is able to communicate with the medical device. The patient can place an RF head 390 of, for example, the emulator 240 near the medical device.

At 5020, the physician selects, for example, a “review” or “interrogate” function in the emulator program. As a result, at 5030, the patient's computer 240 transmits a signal to emulator 240 causing emulator 240 to send a corresponding review request signal to the medical device.

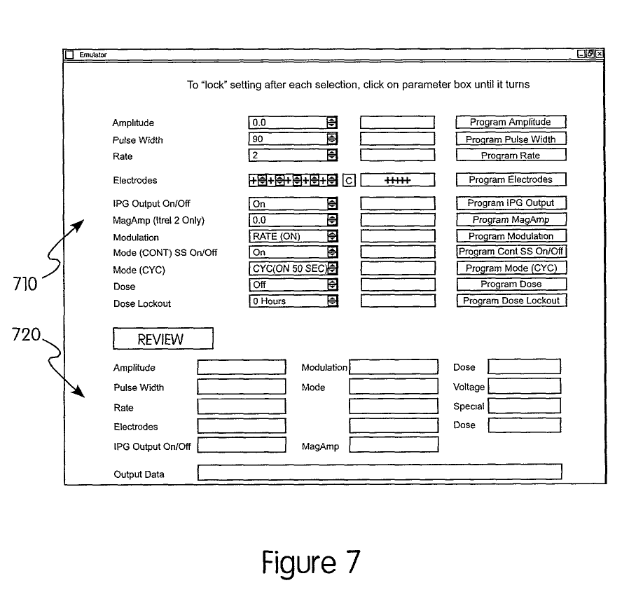

At 5040, the medical device receives the review request signal from emulator 240 and in response transmits the parameter settings to emulator 240. Parameters transmitted to the emulator 240 can include, for example, the pulse width, rate and amplitude of a stimulation signal applied to the patient, and electrode information, such as positive, negative or off. These parameters are conveyed to and processed by the emulator program stored in patient's computer 210 that causes them to be displayed on the physician's computer 220 and/or the patient's computer at 5050. FIG. 7 is an exemplary screen shot of such a display. In portion 710, the variety of settings and possible values for the settings are displayed and can be altered by the physician as desired to tune the medical device. In portion 720, the current values for the various parameters are displayed. Portion 720 can display the settings of the medical device as stated in step 5050 or the settings of the medical device after programming as in step 6040 discussed below. The interactive display can be accomplished through any conventional graphical user interface known in the art.

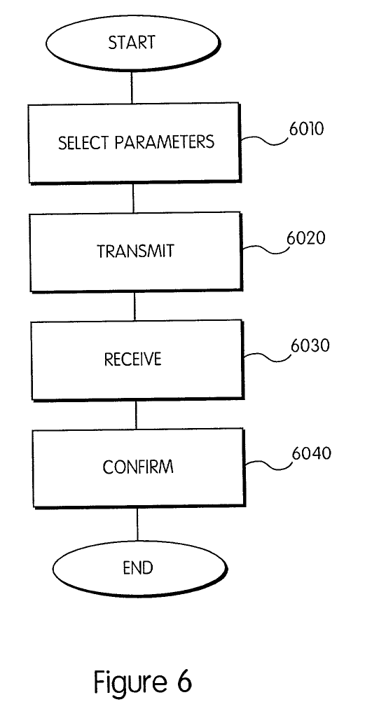

Once the physician receives the settings and diagnoses the patient, the physician can initiate a program sequence to change the settings of the neurostimulator 110. FIG. 6 shows an exemplary embodiment of the program sequence.

For example, at 6010, the physician selects, using the emulator program stored in patient's computer 210, the parameters to be changed. For example, suppose the stimulation signal amplitude should be changed to 0.5 mV. The physician inputs the commands corresponding to this change into the emulator program which, in turn, generates the appropriate control signals for the emulator 240. In response, emulator 240 generates the appropriate RF signals for transmission to the medical device at 6020.

In accordance with an exemplary embodiment, emulator 240 may implement the communication protocol used by the ACTIVA® Tremor Control System, discussed above. In accordance with this protocol, various data values are conveyed by varying the width of an RF pulse. Table 1 shows exemplary pulse widths and the corresponding data value under such a protocol. Note that for a particular instruction (e.g., a change in settings), a predetermined number of RF signals would be sent of varying pulse widths which would be interpreted by the medical device as containing the instruction.

TABLE 1

| Value | Pulse Width (μs) |
|---|---|
| 010000 | 60 |
| 110000 | 90 |
| 001000 | 120 |
| 101000 | 150 |
| 011000 | 180 |
| 111000 | 210 |
| 100100 | 270 |
| 110100 | 330 |
| 101100 | 400 |
| 111100 | 450 |

At 6030, the medical device, such as neurostimulator 110, receives the new settings and adjusts its parameters accordingly.

At 6040, a confirmatory signal can either be automatically or separately requested to be sent from the neurostimulator 110 back to emulator 240. This confirmatory signal conveys the parameters that are now set. These parameters, for example, can be displayed to the physician via a graphical user interface. This confirmation allows the physician to ensure that the requested commands were properly executed.

Additional advantages and modifications will readily occur to those skilled in the art. Therefore, the present invention in its broader aspects is not limited to the specific details and representative devices shown and described herein. Accordingly, various modifications may be made without departing from the spirit or scope of the general inventive concept as defined by the appended claims.

What is claimed is:

1. A system for remotely programming a medical device comprising:
a medical device, the medical device operating on a patient in accordance with one or more settings;
a first computer, the first computer comprising a first communications interface;
an emulator, the emulator being coupled to the first computer and comprising a second communications interface for communicating with the medical device; and
a second computer, the second computer comprising a third communications interface for communicating with the first computer via the first communications interface, wherein the second computer can access the one or more settings of the medical device.

2. The system of claim 1, wherein the medical device is implanted in the patient.

3. The system of claim 1, wherein the first computer comprises a memory, the memory storing a program for accessing the one or more settings of the medical device.

4. The system of claim 3, wherein the program comprises a review procedure for reviewing the one or more settings of the medical device and a programming procedure for modifying the one or more settings of the medical device.

5. The system of claim 1, wherein the second communications interface comprises a radio frequency interface.

6. The system of claim 1, wherein the first computer and second computer communicate via the Internet.

7. The system of claim 1, wherein the first computer and second computer communicate through a dedicated communications link.

8. The system of claim 1, wherein the medical device comprises a neurostimulator.

9. The system of claim 1, wherein the one or more settings include at least one of a stimulation signal pulse width, a stimulation signal rate, a stimulation signal amplitude and an electrode parameter.

10. The system of claim 1 comprising a feedback component coupled to the first computer for capturing a condition of the patient.

11. The system of claim 10, wherein the feedback component including one of a camera and a biosensor.

12. A method of remotely programming a medical device comprising the steps of:
remotely accessing a first computer located proximate a patient by a medical practitioner using a second computer in electronic communication with the first computer;
inputting a change in one or more settings of a medical device implanted in the patient into the second computer;
communicating the change in the one or more settings from the first computer to an emulator connected to the first computer;
transmitting the change in the one or more settings from the emulator to the medical device.

13. The method of claim 12, wherein the step of transmitting comprises transducing a digital signal into an analog signal.

14. The method of claim 13, wherein the analog signal is a radio frequency signal.

15. The method of claim 12, wherein the step of remotely accessing is performed through a remote access program.

16. The method of claim 12, wherein the step of inputting includes initiating a programming sequence.

17. The method of claim 12, comprising performing a review sequence which includes:
selecting a review request at the second computer;
communicating the review request to the emulator via the first computer;
transmitting a review request signal from the emulator to the medical device;
transmitting one or more settings from the medical device to the emulator; and
communicating the one or more settings to the second computer via the first computer.

18. The method of claim 12, wherein the medical device includes a neurostimulator.

19. The method of claim 12, wherein the one or more settings include at least one of a stimulation signal pulse width, a stimulation signal rate, a stimulation signal amplitude and an electrode parameter.

20. The method of claim 12, comprising:
capturing patient feedback at the first computer; and
communicating the patient feedback to the second computer.

21. A system for remotely programming a medical device comprising:
a first computer, the first computer comprising a first communications interface;
an emulator coupled to the first computer and comprising a second communications interface for communicating with a medical device; and
a second computer, the second computer comprising a third communications interface for communicating with the first computer via the first communications interface, wherein the second computer can access one or more settings of the medical device through the emulator.

22. The system according to claim 21, wherein the medical device is an implanted neurostimulator.

23. A system for remotely programming a medical device comprising:
a transceiver component; and
a control component coupled to the transceiver component;
wherein the transceiver component converts control signals from the control component into a programming signal compatible with a medical device, the programming signal generated as a function of input to the control component from a first computer executing a computer executable code, the computer executable code
processing an instruction to change at least one setting of the medical device, the instruction received from a second computer physically remote from the first computer; and
generating the control signal for the medical device, the control signal being transmitted to the control component.

24. The system of claim 23, wherein the computer executable code further includes processing data signals received from the medical device via the transceiver component.

25. The system of claim 23, wherein the transceiver component includes an RF head.

26. The system of claim 23, wherein the control component includes a signal processor, a count generator and a program sequence generator.

27. The system of claim 26, wherein the signal processor amplifies and integrates the electrical signals.

28. The system of claim 26, wherein the count processor generates a plurality of counts using the electrical signal.

29. The system of claim 23, wherein the transceiver component and the control component have an integrated construction.

30. The system of claim 23, wherein the control component is connected to the first computer through one of a serial port, a parallel port and a PCI interface.

31. The system of claim 23, wherein the medical device is an implanted neurostimulator.

32. The system of claim 29, wherein the at least one setting includes one of a stimulation signal pulse width, a stimulation signal rate, a stimulation signal amplitude and an electrode parameter.
